(19) United States
(12) Patent Application Publication
(10) Pub. No.: US 2006/0224034 A1
(43) Pub. Date: Oct. 5, 2006

(54) Radiation shield

(76) Inventor: Kenneth Reever, Hopedale, MA (US)

Correspondence Address: FAY KAPLUN & MARCIN, LLP, 15O BROADWAY, SUITE 702, NEW YORK, NY, 10038, US

(21) Appl. No.: 11/099,863

(22) Filed: Apr. 5, 2005

(51) Int. Cl. A61N 5/00

(52) U.S. Cl. 600/3

(57) ABSTRACT

A radiation shield for insertion into a living body, comprises a compliant member formed of a material which, when formed into a desired shape, substantially retains that shape during insertion into the body, the compliant member including a radiation shielding material and a biocompatible material forming an outer surface thereof.

### BACKGROUND OF THE INVENTION

Electromagnetic and/or nuclear radiation has been used in the treatment of many ailments to destroy diseased tissues. For example, certain cancer cells may be selectively killed as they are more susceptible to damage from radiation of specific energy levels than other types of cells.

Certain illnesses may also be treated by heating a target region of tissue. For example, heat may be used to selectively kill targeted cells taking advantage of an increased susceptibility of these cells to heat or, at lower levels, to increase blood flow to a target region to promote the healing of injured tissue. One method of heating living tissue is through the application of electromagnetic radiation, for example, microwave energy.

A concern common to both ionizing radiation and microwave radiation treatments is to ensure that only the targeted tissue is affected, while leaving the healthy surrounding tissues undamaged. For example, cancerous growths may be intermingled with healthy tissues. In these cases it may be difficult to avoid damage to the healthy tissues as they are subject to the heat and/or radiation directed to the targeted cancerous tissues.

### SUMMARY OF THE INVENTION

In one aspect, the present invention is directed to a radiation shield for insertion into a living body, comprising a compliant member formed of a material which, when formed into a desired shape, substantially retains that shape during insertion into the body, the compliant member including a radiation shielding material and a biocompatible material forming an outer surface thereof.

### BRIEF DESCRIPTION OF THE DRAWINGS

FIG. 1 is a schematic side elevation view of a radiation shield implanted in a patient according to an embodiment of the invention;

FIG. 2 is a top plan view of the radiation shield implanted in a patient shown in FIG. 1; and

FIG. 3 is a perspective cut away view of a radiation shield according to an exemplary embodiment of the invention.

### DETAILED DESCRIPTION

The present invention may be further understood with reference to the following description and the appended drawings, wherein like elements are referred to with the same reference numerals. The present invention is related to radiation shields for preventing injury to non-targeted tissues. In particular, the present invention relates to radiation shields used during treatment of the prostate to protect the patient's bowels from the radiation energy.

As described above, despite healthy tissue's decreased susceptibility to radiation damage, it may still be damaged by radiation applied to treat target cells. Similarly, the heating of target tissue may damage surrounding healthy tissues. Accordingly, embodiments of the present invention may be used to protect the healthy tissue from exposure to radiation and/or heat when nearby tissues are irradiated or heated. Exemplary devices according to the present invention form a shield between target tissue being irradiated and/or heated and non-targeted surrounding tissue (e.g., tissue downstream from a source of radiation). The shield is designed to absorb the energy of the radiation which otherwise would pass into the non-targeted tissues. The shield may be optimized to absorb any or all of nuclear radiation, microwave radiation, or electromagnetic radiation of other frequencies and energy levels.

An example of the application of radiation to treat diseases is radiation treatment of the prostate gland. This therapy may be used to treat cancer of the prostate, and is generally carried out using ionizing radiation. These treatments often cause radiation burns to surrounding organs. In particular, radiation burns to the bowels are common and may cause significant problems later by becoming infected or by turning into fistulas between the rectum and the prostate. Because of the non-sterile nature of the bowel's contents, injuries to the bowels can be difficult to treat and may cause serious problems.

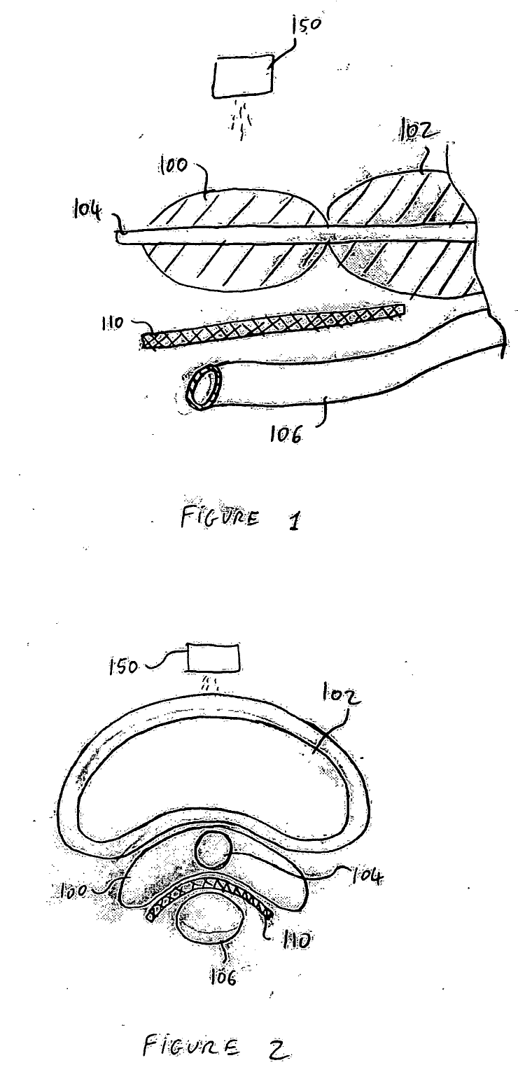

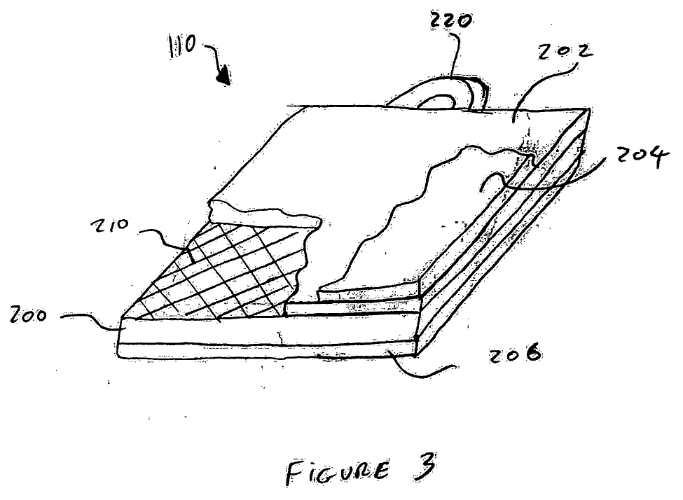

According to exemplary embodiments of the invention, a radiation shield 110 is placed between the prostate and the colon (i.e., in adjacent Denonvilliers' fascia) of a patient before undergoing irradiation of the prostate. FIGS. 1 and 2 show respectively a side elevation and a top plan view of the organs surrounding the prostate 100 during a therapeutic irradiation session. As shown, the radiation shield 110 is placed in the pelvic space that exists between the rectum 106 and the prostate 100. A radiation source 150 is located outside the body requiring that an unshielded path to the prostate 100 be preserved to allow the radiation to reach its target. Alternatively, the radiation source 150 may be introduced through the urethra or perineum and placed in proximity to the prostate 100 to irradiate it from within the body. If transdermal application is carried out in the form of a catheter having a radiation source, additional shielding may be included on the catheter to protect the non targeted parts of the body, and/or to attenuate the radiation.

The radiation shield 110 may be implanted in the patient prior to treatment of the prostate 100 with radiation. In one exemplary embodiment, the radiation shield 110 is implanted transperineally between the prostate 100 and the lower bowel, i.e. the rectum 106, to protect the latter from the radiation. The radiation shield 110 may be removed from the patient after the treatment has been completed to prevent ongoing discomfort to the patient. However, as will be described below, the radiation shield 110 is preferably made from a flexible, compliant, biocompatible material with a radiation shielding layer 200 formed therein. The flexibility of the material allows patient discomfort associated with placement of the radiation shield 110 in the abdomen to be minimized. Accordingly, in one embodiment the radiation shield 110 may be left in place within the patient after completion of the irradiation, to avoid the discomfort and increased risks associated with the additional surgery needed to remove the shield.

The exemplary radiation shield 110 may be sufficiently flexible so that it may be molded into a desired shape by the surgeon prior to insertion in the patient. For example, the radiation shield 110 may be molded into a curvature approximating the shape of the prostate 100 and the rectum 106 so that the maximum shielding from the radiation can be obtained with minimal discomfort to the patient. The material of the radiation shield 110 is preferably also sufficiently compliant to prevent the desired shape from being changed during insertion. The material may be selected to resist a specific force before deforming, depending on the medical procedure for which the device is used. Alternatively, the radiation shield 110 may be pre-shaped during manufacture, and various shapes and sizes may be provided to the surgeon to fit a variety of different patients. In general, the radiation shield 110 is preferably sufficiently flexible to accommodate normal movement of the patient without being displaced from its desired position and without undue discomfort to the patient. This is particularly important in the case where the radiation shield 110 is left within the patient after the treatment has been completed.

FIG. 3 shows an exemplary cut away perspective view of the radiation shield 110. The material of which the radiation shield 110 is made is selected for its ability to block a type of radiation to which the target tissue is to be subjected. For example, when ionizing nuclear radiation is to be used, lead, gold, tantalum, tungsten, bismuth, silver or platinum may be used as shielding materials. However, additional materials that can absorb or shield from nuclear radiation may be used equally effectively and the above list is intended to be exemplary only. Mixtures and alloys of these and other materials may also be used effectively to form the radiation shield 110.

In the exemplary embodiment, the shielding layer 200 of the radiation shield 110 contains radiation absorbing material or materials. Since the shielding layer 200 is preferably flexible, various construction methods may be employed to achieve a radiation shield 110 having the desired material properties. For example, a semi-flexible woven or knitted material may be formed from strands 210 of the radiation shielding material. Alternatively, a gauze-like construction may be implemented using a fabric constructed from the radiation shielding materials. In a different embodiment, the shielding layer 200 may be formed of a semi rigid foil which may be shaped as required, and which possesses the desired flexibility and other mechanical properties. Many radiation shielding materials may be worked into threads or foils, which may be directly used to construct the shielding layer 200 as described above.

In a different embodiment, a composite material may be formed using a flexible matrix seeded with the radiation shielding material. This approach may be preferred in cases where the radiation absorbing/shielding material does not have mechanical properties suitable to form a flexible shield. For example, a polymeric or textile matrix having sponge-like or gauze-like mechanical properties may be seeded with one or more of the above mentioned materials. In this exemplary embodiment, the strands 210 forming the shielding layer 200 may be made of the matrix material. A suitable radiation shielding material may then be seeded within the matrix material, to confer the radiation protection properties. Alternatively, a mixture of a polymeric material containing the radiation shielding metals may also be used, to obtain a semi-flexible shield which can be easily shaped to fit within the patient's body, following the curvature of the relevant organs.

Some construction details of the radiation shield 110 may also be dictated by the type of radiation to be used in treating the patient and which is to be blocked by the radiation shield 110. For example as described above, different wavelengths and energy levels of electromagnetic radiation may be used to irradiate target tissue including, for example, x-rays, gamma-rays and heat in the form of infrared or microwave radiation. It will be apparent to those of skill in the art that different radiation shielding materials may be used to protect the nearby organs from irradiation depending on the characteristics of the radiation. In the case of microwave radiation, the shielding layer 200 may comprise a metallic grid having an aperture selected to interfere with the propagation therethrough of microwave radiation of a given wavelength. Alternatively, any other appropriate microwave shielding method known in the art may be applied to the radiation shield 110 to protect surrounding organs from excessive heating. As described above, the mechanical properties of the radiation shield 110 permit it to be shaped as desired and to retain the desired shape while retaining a degree of flexibility after implantation.

According to exemplary embodiments of the invention, the radiation shield 110 may also be encased, laminated or coated with a bio-compatible material. The radiation shield 110 is implanted in the patient's body, and in some cases is designed to remain in the patient permanently. Accordingly, it is important to prevent or minimize adverse reactions of the body to the material of the radiation shield 110. As shown in FIG. 3, a layer of bio-compatible material 204 may form an outer surface of the radiation shield 110. The bio-compatible material may, for example, be one of PTFE, polyethylene, ethylene vinyl acetate (EVA), silicone polycarbonate, titanium, nickel-titanium alloys, tantalum or stainless steel. In addition to these materials, it will be apparent to those of skill in the art that other bio-compatible materials may be used to coat the radiation shield 110.

An optional layer 202, also shown in FIG. 3, may be applied to the radiation shield 110 in another embodiment of the invention. The layer 202 may comprise a thermal barrier material adapted to protect the surrounding organs from damage due to heating. For example, the layer 202 may be adapted to protect the patient's bowels from heating due to the conduction and/or convection of heat from the prostate as the prostate 100 is subject to thermal treatment. The layer 202 may be formed of any of a variety of known flexible, heat insulating materials. In an exemplary embodiment, the thermal shielding function of the optional layer 202 may be combined within the bio-compatible layer 204, which in that case provides a coating or casing to the shielding layer 200 that is both bio-compatible and thermally insulating. A separate layer 202 is therefore not necessary in this case.

To properly protect the surrounding organs from damage due to radiation, the radiation shield 110 is designed to remain in place for the duration of the treatment. To prevent unwanted migration of the radiation shield 110, anchoring devices may be included to secure it in place. For example, one or more suture tabs or clamps may be provided, so that the surgeon may affix the radiation shield 110 to nearby tissues thereby preventing displacement of the radiation shield 110 from a desired position, for example, while moving the patient. In one exemplary embodiment shown in FIG. 3, a plurality of suture tabs 220 is provided on a periphery of the radiation shield 110. After the radiation shield 110 has been inserted in the patient, for example between the prostate 100 and the rectum 106, the surgeon may suture the radiation shield 110 to the surrounding tissue immobilizing it relative to the adjacent organs. Radiation or heat treatments may then be administered with confidence that the surrounding organs (such as the rectum 106) are protected from damage due to the treatment.

The present invention has been described with reference to specific embodiments, and more specifically to a radiation shield for ionizing and microwave radiation used to treat prostate cancer. However, other embodiments may be devised that are applicable to other types of cancers and other organs, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A radiation shield for insertion into a living body, comprising:
a compliant member adapted to be formed in a desired shape;
a radiation shielding material of the compliant member; and
a biocompatible material forming an outer surface of the compliant member.

2. The radiation shield according to claim 1, wherein the compliant member is formed of a material which, when formed into a desired shape, substantially retains that shape during insertion into the body.

3. The radiation shield according to claim 1, further comprising a heat insulating portion of the compliant member.

4. The radiation shield according to claim 3, wherein the heat insulating portion is adapted to limit at least one of conduction and radiation heat transfer through the compliant member.

5. The radiation shield according to claim 1, further comprising an ionizing radiation shielding layer of the radiation shielding material.

6. The radiation shield according to claim 5, wherein the ionizing radiation shielding layer comprises a high radiation density material to shield from nuclear radiation.

7. The radiation shield according to claim 5, wherein the ionizing radiation shielding layer comprises at least one of lead, gold, tantalum, tungsten, bismuth, silver and platinum.

8. The radiation shield according to claim 1, further comprising a microwave radiation shielding layer of the radiation shielding material.

9. The radiation shield according to claim 8, wherein the microwave radiation shielding layer comprises a grid-like element having grid cells sized to shield from a selected range of microwave radiation wavelengths.

10. The radiation shield according to claim 1, wherein the biocompatible outer portion comprises one of a cladding material or a coating material.

11. The radiation shield according to claim 10, wherein the biocompatible outer portion is formed of at least one of silicone, PTFE, polyethylene, ethylene vinyl acetate, polycarbonate, titanium, nickel-titanium alloys, tantalum and stainless steel.

12. The radiation shield according to claim 1, wherein the biocompatible material comprises a heat insulating material.

13. The radiation shield according to claim 1, wherein the compliant member comprises one of a semi flexible woven material, knitted material, gauze-like material and semi rigid foil material.

14. The radiation shield according to claim 1, further comprising an anchoring portion for binding the shield to an anatomical structure so that a relative position of the shield and the anatomical structure is maintained.

15. A method for radiation treatment comprising:
implanting a radiation shield between a target tissue to which radiation is to be directed and a surrounding non-targeted tissue, the radiation shield being formed of a material adapted to prevent the radiation to be applied from passing therethrough; and
irradiating the target tissue from a source located such that the radiation shield is disposed between the source and the surrounding tissue.

16. The method according to claim 15, further comprising removing the implanted radiation shield after completion of the radiation treatment.

17. The method according to claim 15, further comprising anchoring the radiation shield to an anatomical structure to maintain a position of the radiation shield relative to the anatomical structure.

18. The method according to claim 15, wherein the implanting step comprises implanting the radiation shield transperineally between a patient's prostate and colon.

19. The method according to claim 15, further comprising leaving the implanted radiation shield between the target tissue and the surrounding non-target tissue after the irradiating is completed.

20. A tissue protector for implantation in a body adjacent to tissue targeted for at least one of radiation and heat treatment, the tissue protector comprising:
a radiation shield formed of a material preventing the radiation to be applied from passing therethrough;
a heat insulating component adapted to block a transfer of heat therethrough; and
a bio-compatible component surrounding the radiation shield and the heat insulating component.

21. The tissue protector according to claim 20, further comprising an anchoring element adapted to be bound to an anatomical structure to maintain the tissue protector in a desired position relative to the anatomical structure.

22. The system according to claim 20, wherein the implantable radiation shield is optimized to shield from ionizing radiation.

23. The system according to claim 20, wherein the implantable radiation shield is optimized to shield from microwave radiation.

24. The system according to claim 20, wherein the heat insulating component and the bio-compatible component are combined in a single layer.

25. The system according to claim 24, wherein the single layer is one of a coating and a cladding surrounding the radiation shield.

26. The system according to claim 20, wherein the radiation shield comprises a substantially planar element formed of a compliant material which may be bent into a desired shape and which substantially maintains the desired shape during insertion into the body.

27. The system according to claim 20, wherein the radiation shield comprises a substantially planar element shaped to fit in a preselected anatomical location.

28. A radiation treatment device comprising:
a radiation shield implantable between a target tissue to which radiation is to be directed and a surrounding non-targeted tissue; and
a component of the radiation shield adapted to prevent the radiation to be applied from passing therethrough.

29. The radiation treatment device according to claim 28, further comprising an outer surface portion of the radiation shield formed of a biocompatible material.

30. The radiation treatment device according to claim 28, wherein the radiation shield is formed of a compliant material adapted for shaping before being implanted.
